(19) United States
(12) Patent Application Publication
(10) Pub. No.: US 2022/0319634 A1
(43) Pub. Date: Oct. 6, 2022

(54) Methods and systems for assessing drug development outcomes

(71) Applicant: Julian M. Kleber, Berlin (DE)

(72) Inventor: Julian M. Kleber, Berlin (DE)

(21) Appl. No.: 17/223,038

(22) Filed: Apr. 6, 2021

(51) Int. Cl. G16B 5/20; G16B 40/00; G16B 15/30; G16C 20/50; G16C 20/70

(52) U.S. Cl. CPC G16B 5/20; G16B 40/00; G16B 15/30; G16C 20/50; G16C 20/70

(57) ABSTRACT

Systems and methods are disclosed herein for computer-aided method utilizing machine learning, artificial intelligence and automated docking for developing, customizing, discovery and maintaining the process of drug development pipeline finding of compounds containing Boron and Nitrogen, symmetric, aromatic, heteroaromatic, cyclic, heterocyclic compounds for drugs. The proposed method uses and identifies Boron Nitrogen Organic Compounds as a drug candidate for drugs through the use of software. The system works by automatically processing data to identify potential compounds containing Boron Nitrogen organic compounds as drug candidates using machine learning. The system further provides molecular data as smiles/inchi/ calculates properties and predicts the pharmaceutical activity with machine learning algorithms. It further provides functionality of automated docking to novel 3D protein structures and automated structure generation using RNN (recurrent neural networks) or LSTM (long short-term memory). The system can present neural networks and LSTM (long short-term memory) networks to the user.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever

### BACKGROUND

### Field of the Invention

The present invention relates generally to the field of system and method for drug development pipeline process and in particular relates to methods and systems for automated iterative drug discovery providing findings and discovery of novel lead structures, cyclic, polycyclic, metallo-organic, symmetric, aromatic, heteroaromatic, drugs and novel drug candidates containing boron nitrogen compounds.

### Description of the Related Art

Current methods of drug discovery known in the art suffer from many complications in required materials, speed, cost, difficulty and the like. For example, one such known method relies on a combinatorial chemical library or the members of a directed diversity chemical library.

A combinatorial chemical library is a prechosen plurality of compounds manufactured simultaneously as a mixture. This plurality will have a common structural core and each member will represent a unique configuration of substitution at specific positions on the common core. Most commonly the common core will be attached to a bead structure to facilitate handling. The strategy for preparation is usually one which will lead to a mixture of all possible compound types but any bead will only bear one type of compound. To facilitate identification the compound or the bead or a linker group may contain a coding tag to aid identification. Alternatively, identification may be achieved by mass spectral analysis of the compound after cleavage from the bead. The preparation of a bead-bound (or a solution mixture) combinatorial library is essentially a manual process and for the duration of the library synthesis probably represents the highest level of manual productivity of a chemist. However, library production must be preceded by months of exploratory chemistry to achieve the near perfect yield of each compound required to ensure that unplanned artifacts are not included in the library, then succeeded by a long period of quality assurance to ensure screening results are not misleading. Because of the very limited range of chemistry that can be carried out on solid-supports and the fact that successful library production requires that all members must be synthesizable under the same reaction conditions, it is difficult to use a combinatorial library to create diverse structures. Because of the variable effectiveness of compound cleavage routines, assay concentration is uncertain. In addition, a single concentration does not allow ranking of compound and therefore structure-activity relationships cannot be developed. Essentially, a combinatorial library must be looked on as a slowly produced compound set that gives very limited information on screening. As such, only with great difficulty can it be used in a closed loop manner (i.e. assay results are used to inform the design of a new generation of compounds), and the response cycle time will be impossibly protracted (months to years). The combinatorial library is more a tool for serendipitous discovery of active compounds and even here it represents an unjustifiably dense representation of a minute fraction of chemical space. A directed diversity library is a prechosen plurality of chemical compounds which are formed by selectively combining a particular set of building blocks in separate reactors. This obviates the need for near perfect yields since individual reaction products can be subjected to individual purification. In addition, quality control and assay issues are eased and the concentration dependence of activity or affinity can be produced to enable the ranking of compound properties. In essence this would be the procedure that a chemist would perform to manually synthesize a compound. The feature here is that only one reaction sequence is used and the differences in the library members arise from differences in the non-common portion of the reagents. A plurality of compounds can therefore be produced more quickly than if each library member required a new route to be explored and optimized for production. The disadvantage is the limit to the diversity of compounds that can be produced by a single route and the large amount of time required (relative to the combinatorial approach) for handling compound preparation and purification on an individual basis. Efficiency is only gained when the library can be prepared in batch mode (parallel processing) usually by employing automation. Therefore, the frequency at which structure-activity relationships can be updated is determined by batch size (usually hundreds into thousands to offset the overheads of automating the process) and the associated cycle time for designing, preparing, purifying, registering, transporting, assaying and reporting the data for the batch (usually months). Because of the specialized nature of high throughput chemical synthesis and high throughput biological screening there is a perceived need in pharmaceutical research and development to logically divide responsibilities by discipline in order to maintain core competences and develop expertise in a collegiate fashion. Because of their large capital requirement, there is also a need to provide these services through one or very few “centralized” facilities. There is a perceived need also to physically divide activities on the basis of their different resource demands. For example, a chemistry department, its accommodation and equipment, is quite different from a department conducting biological research, and each differs from an information technology department. This practice of division of responsibilities coupled with high throughput technology which relies on adherence to batch processing in accordance with standard operating procedures, frequently has a potentially adverse effect: the different departments become inflexible enterprises in their own right with their own goals and the bigger they become, the more disconnected they become from other enterprises essential to the task of drug discovery. Disconnection can be both physical and temporal. Large batches of compounds from a chemistry center can end up being transported large distances to a screening center and the relative scheduling of the preparation and screening events of batched compounds is sub-optimal in respect of maximizing the use of biological data to inform compound design. Thus, whilst high throughput chemistry groups may achieve high productivity in terms of compounds produced per chemist, and high throughput screening groups achieve a high number of assays run per staff member, there generally is less real-time interaction and feedback between these two activity silos than usually can be found between adjacent groups in full communication, performing these missions manually and at low throughput. However, low throughput groups incur a time and cost penalty on the enterprise focused on generating new drugs. Thus, current large pharmaceutical research and development in practice seeks a balance set towards high throughput technologies for the early lead discovery stages and graded to low throughput synthesis and assay as lead optimization approaches a clinical candidate.

Apart from the organizational difficulties, reduced interaction and feedback also arises from the nature of current high throughput methods which are based on the numerical efficiencies derived from working in very large batches in a parallel manner. Thus, parallel synthesis in chemistry requires the validation of only one reaction route to prepare many compounds which will often share common structure to a substantial degree. In high throughput screening the time taken to validate the high throughput assay is recouped by its repeated high-speed use across many plates of compounds. Unfortunately, this dependence on large batches to deliver numerical efficiency provides no opportunity for iterative improvement against the criteria set for a successful drug candidate. An additional barrier is set by processes designed to deal with the practical reality that synthesized and/or assayed compounds have to be physically moved from the site of preparation to the site of assay. These include isolating solid single materials, bottling, labeling, registering, storing, retrieving from store, dispensing, re-dissolving, and distributing. These processes require that sufficient amounts of compounds are prepared to allow these processes to be physically possible. Often several hundred milligrams are required to satisfy the storage and retrieval demands and transmission wastage, yet many modern assays require no more than a few thousand molecules. Indeed, there is much extra to be gained in information content from assays conducted on a ‘single molecule’ scale as there is a clarity with regard to signal source, there is evidence of mechanism and the information is not obscured by aphasic information from a plurality of molecules at different stages of action. In addition, there are inconvenient waiting times involved in many of these processes, particularly if the chemistry, screening, and compound management groups are physically remote.

Other methods known in the art include manual or semi-manual chemical reaction optimization as it is routinely practiced. Manual or semi-manual iterative medicinal chemistry requires human intervention, and is very slow. For efficiency in respect of time or cost, the process is usually performed through the construction of combinatorial libraries or parallel synthesized arrays conducted in wells or flasks following a pre-conceived experimental protocol designed to test the influence of pre-decided parameters exemplified through sets of compounds in which the strength of the parameter is varied. Manual or semi-manual iterative medicinal chemistry usually requires substantial human intervention but is a very slow process involving the activities of several different knowledge disciplines which may be located at significant distances from one other. However, it should be noted that stepwise iteration using the accumulating data to inform the design of the next single compound, represents the most powerful search method and demands the fewest chemical examples to explore the greatest amount of chemical diversity space. Another commonly practiced method known in the art is the sequential use of automated high throughput chemistry and automated high throughput screening. In automated high throughput chemistry, a plurality of compounds with a familial relationship is prepared according to a standard method and placed in a compound store. In automated high throughput screening a plurality of diverse compounds drawn from a compound store is screened against a single biological target by a standardized method. In the de novo lead iteration of the present invention, by contrast, the products of a single reaction are assayed directly as single entities in one or more assays to gain information. The information is used to predict the structure of a subsequent compound with improved properties which need not have a familial relationship with the original compound nor be created through a cognate synthetic sequence.

The medicinal chemistry platform as deployed in the pharmaceutical industry is a virtual paradigm and encompasses work carried out by different disciplines usually located in different locations where the separation of the physical activities of chemical compound creation and biological assay are carried out at locations separated by more than 3 meters and separated by at least one wall.

“Originally a scientific curiosity of physicists and chemists, microfluidics now appears ready to transform traditional assay systems in academia and biotech as well as in big pharma and hospitals, with devices labeled as ‘pinhead Petri dishes’ and ‘Lab-on-a-chip’.” Clayton, Nature Methods 2, 621-627 (2005). Microfluidic devices have been known in the art for only a few years, beginning primarily with such lab-on-a-chip devices that require samples to be introduced into the device in a highly specific form, such as premixed in a homogenous reagent mixture. A review in 2003 concluded that while many microfluidic devices were in active development, integration of all laboratory functions on a chip, though the commercialization of truly hand-held, easy to use microfluidic instruments has yet to be fulfilled. Weigl, Advanced Drug Delivery Reviews, 55 (2003) 349-377, specifically incorporated herein by reference in its entirety. See also Fletcher et al, Tetrahedron 58 (2002) 4735-4757. However, advances in microfluidics have brought the integration of microfluidic and electronic components, as for example disclosed in U.S. Pat. No. 6,632,400.

Additional discussion of microfluidic chemistry may be found in Fletcher et al., Lab Chip
(2002) 2:102-112; Fletcher et al., Lab on a Chip (2001) 1:115-121; Watts et al., Chem. Soc. Rev.
(2005) 34:235-246; Broadwell et al., Lab on a Chip (2001) 1:66-71; Kikutani et al., Lab Chip
(2002) 2:188-192; Skelton et al., Analyst (2001) 126:11-13; Haswell et al., Chem. Commun. (2001) 391-398; Wong Hawkes et al., QSAR Comb. Sci (2005) 24:712-721.
U.S. Pat. No. 6,391,622 discloses integrated systems performing a wide variety of assays and other fluid operations on a micro scale.
International Patent Application Pub. WO 2004/089533 discloses microfluidic systems.
U.S. Pat. No. 5,463,564 discloses an iterative synthesis system based on directed diversity chemical libraries.

Looking at the prior art there are no advancements that have been seen in similar regards which are not only convenient to masses but also contribution toward society and environment. Therefore, it would be advantageous to have an improved method, apparatus, and computer instructions for providing user an online platform where they can by using and identifying boron nitrogen organic compounds as drug candidates for drugs by using the software which works on machine learning and artificial intelligence. The proposed method helps not only to assess the output but also facilitates the discovery of new lead structures, drugs and drug candidates.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, and abstract as a whole.

It is therefore the purpose of the invention to alleviate at least to some extent one or more of the aforementioned problems of the prior art and/or to provide the relevant public with a suitable alternative thereto having relative advantages.

The primary object of the invention is related to the provision of an improved online system which is a drug development and discovery pipeline finding specifically for Boron Nitrogen Compounds for drugs.

It is further the objective of the invention to provide a method, apparatus, and computer instructions for providing a platform which understands, identify and store the information based on machine learning.

It is also the objective of system to provide a method whereby the system works by automatically processing data to identify potential compounds containing boron nitrogen organic compounds as drug candidates using machine learning.

It is also the objective of the invention to provide a platform where user can access the data and download or read the data.

It is further the objective of the invention to provide a level of interaction and quick access to users allowing to share the generated output.

It is also the objective of invention to provide fast disaster response, fast pandemic response and benign process.

It is moreover the objective of the invention to provide an application which gets molecular data as smiles/inchi/ calculates properties and predicts the pharmaceutical activity with machine learning algorithms.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The following description are illustrative and are not to be construed as limiting. Numerous specific details are described to provide a thorough understanding. However, in certain instances, well known or conventional details are not described in order to avoid obscuring the description. References to one or an embodiment in the present disclosure are not necessarily references to the same embodiment; and, such references mean at least one.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but no other embodiments.

One embodiment of the present invention provides a method in which the application of a first analysis stage is used for initial screening of individual discriminating variables included in the solution. Following initial individual discriminating variable selection, subsets of selected individual discriminating variables are found particularly boron nitrogen organic compounds, through use of a second discriminatory analysis stage, to form a plurality of intermediate combined classifiers.

Once determined from the training dataset, the selected individual discriminating variables, each of the intermediate combined classifiers, and the single meta classifier can be used to discern or clarify relationships between subjects in the training dataset and to provide similar information about data from subjects not in the training dataset.

In typical embodiments, each element of the solution subspace is completely sampled by artificial intelligence process. An initial screen is performed during which each variable is sampled.

In the present invention, straightforward artificial intelligence techniques are utilized in order to reduce computational intensity and reduce time. There are no iterative processes or large exhaustive combinatorial searches inherent in the systems and methods of the present invention that would require convergence to a final solution with an unknown time requirement. Given a priori knowledge of the number and type of multivariate data used for training, the computational burden and memory requirements of the systems and methods of the present invention can be fully characterized prior to implementation.

As new training data becomes available; the systems and methods of the present invention allow for the incorporation of such data into the meta classifier and the direct use of such data in classifying subjects not in the training population. In other words, when new information becomes available, the systems and methods of the present invention can immediately incorporate such information into the diagnostic solution and begin using the new information to help classify other unknowns.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A method for characterizing the probability of a clinical outcome of a subject based on machine learning, simulations and artificial intelligence, comprising:
a. constructing a probability space defined by a set of discrete clinical outcomes, each of which is characterized by a statistical distribution of at least one biological marker which can be boron and nitrogen, symmetric, aromatic, heteroaromatic, cyclic and heterocyclic compounds;
b. obtaining subject data corresponding to the at least one biological marker;
c. obtaining data related to borazine symmetric heteroaromatic compounds;
d. calculating the position of said subject data in said probability space, thereby characterizing the probability of the clinical outcome of said subject;
e. Presenting symmetric lead generation and derived compounds with broken symmetry keeping the symmetric core;
f. presenting automated system for docking to novel 3D protein structures and automated structure generation using RNN;
g. presenting graph based neural networks and LSTM (long short-term memory networks)
h. calculating molecular data as smiles/canonical smiles/inchi/pdb/xyz calculates properties and predicts the pharmaceutical activity with machine learning algorithms
i. generating novel lead structures not present in current databases are achieved via reinforcement learning methods and RNN or LSTM networks.
